(19) United States
(12) Patent Application Publication
(10) Pub. No.: US 2007/0276195 A1
(43) Pub. Date: Nov. 29, 2007

(54) Systems and methods for wound area management

(75) Inventors: Tianning Xu, San Antonio, TX (US); Jonathan Paul Jaeb, Boerne, TX (US)

Correspondence Address: LEGAL DEPARTMENT INTELLECTUAL PROPERTY;KINETIC CONCEPTS, INC., P.O. BOX 659508, SAN ANTONIO, TX, 78265-9508, US

(73) Assignee: KCI Licensing, Inc., San Antonio, TX (US)

(21) Appl. No.: 11/433,817

(22) Filed: May 12, 2006

(51) Int. Cl. A61B 5/00

(52) U.S. Cl. 600/300

(57) ABSTRACT

Systems and methods for capturing and digitizing an image of a wound and/or a wound trace from a patient and determining there from a degree of change in the characteristics of the wound. A first embodiment includes a transparent/translucent film onto which a dark outline of the wound is traced. The film is fixed to a reference template that provides a geometrically defined reference area. The film/template assembly is imaged with a digital imaging device associated with a handheld digital processor (such as a PDA). The digital image of the template and the wound trace are analyzed to identify the wound tracing and quantify the area within the closed curve. A second embodiment includes imaging the wound site with a reference tag and viewing the image on a display with an associated graphical data input device. A trace of the wound perimeter is made on display with the graphical data input device to establish a data set for the wound perimeter. The data set for the wound trace and the reference tag are analyzed to identify and quantify the wound area. In each embodiment, the system includes a display for providing both a view of the wound trace and the calculated data associated with the wound area.

### BACKGROUND OF THE INVENTION

1. Field of the Invention

The present invention relates to generally to systems and methods for measuring a rate of biological tissue healing. The present invention relates more specifically to systems and methods for capturing, digitizing, and analyzing an image of a wound and determining there from a degree of change in the characteristics of the wound.

2. Description of the Related Art

Many advances have recently been made in the field of wound therapy that have greatly increased the rate and quality of the wound healing process. Commensurate with providing an effective wound therapy regimen is the ability to make measurements of the size of the wound and the rate at which it heals. One coarse but generally effective manner of determining the rate of healing for a wound is to track changes in the overall wound size over time.

Previous efforts to measure and track changes in the size of a wound have failed in many respects to provide the necessary information to health care providers to allow an assessment of the efficacy of a therapy. A number of existing methods for measuring the size of a wound involve the use of a transparent or translucent film and a pen or marker to trace the patient's wound along its edge and then digitize the trace in some manner for analysis. One example of this approach involves placing the film with the trace on a touch-pad surface and re-tracing the outline of the wound. The touch-pad electronic instrumentation translates the trace into a digital array of data that may then be analyzed. A processor associated with the electronic instrumentation then calculates the area inside the trace. Since no scaling of the trace occurs the wound size measurable with such systems is limited to the size of the instrument's touch sensitive surface. In addition, such systems involve two tracings, one on the patient and then a second on the touch pad, a process that is susceptible to progressive errors and inaccuracies.

Other systems known in the art rely upon a direct digital imaging approach that takes into consideration the distance and angles associated with the image capture. These systems tend to be highly complex and to require significantly greater processing capabilities to take into account variations in the angles and distances associated with the imaging view. In the end, even these complex systems fail because image recognition processes are often unable to accurately and consistently define a wound perimeter.

### Background on Wounds and Wound Healing Processes

A wound is generally defined as a break in the epithelial integrity of the skin. Such an injury, however, may be much deeper, including the dermis, subcutaneous fat, fascia, muscle, and even bone. Proper wound healing is a highly complex, dynamic, and coordinated series of steps leading to tissue repair. Acute wound healing is a dynamic process involving both resident and migratory cell populations acting in a coordinated manner within the extra-cellular matrix environment to repair the injured tissues. Some wounds fail to heal in this manner (for a variety of reasons) and may be referred to as chronic wounds.

Following tissue injury, the coordinated healing of a wound will typically involve four overlapping but well-defined phases: hemostasis, inflammation, proliferation, and remodeling. Hemostasis involves the first steps in wound response and repair which are bleeding, coagulation, and platelet and complement activation. Inflammation peaks near the end of the first day. Cell proliferation occurs over the next 7-30 days and involves the time period over which wound area measurements may be of most benefit. During this time fibroplasia, angiogenesis, re-epithelialization, and extra-cellular matrix synthesis occur. The initial collagen formation in a wound will typically peak in approximately 7 days. The wound re-epithelialization occurs in about 48 hours under optimal conditions, at which time the wound may be completely sealed. A healing wound may have 15% to 20% of full tensile strength at 3 weeks and 60% of full strength at 4 months. After the first month, a degradation and remodeling stage begins, wherein cellularity and vascularity decrease and tensile strength increases. Formation of a mature scar often requires 6 to 12 months.

### Efforts in the Related Art to Measure Wound Healing Processes

Because wound treatment can be costly in both materials and professional care time, a treatment that is based on an accurate assessment of the wound and the wound healing process can be essential. Current problems in the prior art include imperfect methods for actually measuring (directly or indirectly) the size of the wound. Clearly, the ideal measuring instrument would be dimensionally accurate, reliable, provide data for a permanent record, and provide for the accurate discrimination of wound versus periwound areas. It should be capable of measuring a wound of any size or shape in any location on the body. Those parts of the system that are directly associated with the patient should be portable and made of inert material. They must be utilized with minimum patient discomfort, and should not introduce contamination into the wound. Additionally, the instrumentation associated with “translating” the wound image into a measurable form should be cost effective and should not require excessive training for routine clinical use.

Obtaining consistent wound measurements is also an important factor in accurately determining changes in wound size. Different practitioners will typically be involved in taking the wound measurements for a particular patient, so accurately reproducible techniques should be used in order to produce results that are relevant, accurate, unbiased, and efficient. The optimal measurement device would have consistency between caregivers and have minimal variation resulting from patient positioning, wound stretching, or other changes that would affect both variance and reliability (for both intra-rater and inter-rater concerns).

The frequency of assessment of a wound is often based on the wound characteristics observed at a previous stage in the healing process or is simply carried out according to the health care provider's orders. The effectiveness of the prescribed interventions cannot be evaluated unless baseline assessment data can be compared with the follow up data. Thus, the consistency of measurements from one observation period to the next is crucial.

The definition of a completely healed wound is sometimes stated as being a wound that has totally re-epithelialized and stays healed for a minimum of 28 consecutive days. Generally, wound healing proceeds through an orderly repair process, so certain parameters such as the size and shape of the wound, the rate of the healing, and the status of the wound bed are appropriate markers for assessing progress through this process. For chronic wounds, this may not occur due to complex and non-uniform healing processes. Complete wound closure may not be achieved nor be a realistic objective endpoint for judging the outcome for certain chronic wounds.

In addition to the systems described above that measure the two-dimensional area of a wound, various methods also exist for measuring wound volumes that extend below the surface of the skin. Common wound volume measurement techniques include molds, fluid installations, caliper devices, and stereophotogrammetry. These techniques all, however, suffer from various problems with accuracy, repeatability, or complexity. A wound mold, for example, although it provides a highly reliable measurement, is messy and time consuming, uncomfortable, and risks contaminating the wound.

Another method to estimate the size of the wound is the installation of saline into the wound covered by a sheet or film. The fluid is then extracted and measured to determine a volume. However, this fluid technique is imprecise, can be messy, and is often difficult to carry out. The wound can also be contaminated with such approaches. Caliper based system use plastic coated disposable gauges that rely upon a three dimensional coordinate system to measure the wound volume directly. This approach uses a mathematical formula to calculate the volume but suffers frequently from technique variations in the acquisition of the data.

Stereophotogrammetry systems typically use a video camera attached to a computer or other microprocessor based device. In a stereophotogrammetric system for wound measurement, the clinician places a target plate in the principle plane of focus adjacent to the wound and captures the combined image on video tape. A cotton-tipped applicator is used to mark the wound depth at the deepest point. After the image is captured, the clinician uses the computer to trace the length and width of the wound. The length of the cotton-tipped applicator is also measured and recorded as the depth. The images are then stored on the computer for later use, analysis and comparison. Stereophotogrammetric systems often provide accurate and reproducible measurements of wound size and volume but do so at great expense and complexity.

One effort in the field to note is described in U.S. Pat. No. 5,967,979 issued to Taylor et al. on Oct. 19, 1999 entitled Method and Apparatus for Photogrammetric Assessment of Biological Tissue. This patent describes a remote wound assessment method and apparatus that includes forming an oblique image of both the wound and a target plate containing a rectangle that is placed near the wound. Coordinate transformations allow measurement of both the size of the wound and its contours. Producing two separate images at different oblique angles results in the three dimensional features of the wound being measurable.

Efforts in the past involving indirect wound measurements (i.e. transferring an outline trace of a wound to some digitizing device) have suffered in part from the simple need to create a second tracing in order to transfer the wound image to instrumentation suitable for making measurements. Such systems were typically limited in size by the template used or by the touch sensitive surface utilized with the instrumentation. In addition, many of the imaging methods previously used do not work well on a wound that wraps around a limb or is otherwise not in a plane parallel to the CCD array plane of the imaging device.

### SUMMARY OF THE INVENTION

It would therefore be desirable to have a wound measurement system that achieved all of the goals mentioned above, namely; accuracy, discrimination (the ability to distinguish the wound area from the periwound area), repeatability, non-invasiveness, simplicity, and cost effectiveness. Those parts of the system that might come in direct contact with the patient should be aseptic and disposable. The processing components of the system should be straightforward and intuitive to use by modestly skilled clinicians. The processing components should likewise be capable of providing historical data to allow the user to track changes over time.

The systems of the present invention take advantage of ever more available and inexpensive digital imaging tools while at the same time recognizing that the most accurate discrimination tool is still the eye of the clinician. A first preferred embodiment of the present invention utilizes a transparent or translucent film (containing the wound trace); a background/template (comprising, for example, a half rigid board with visually contrasting background and reference surface areas, such as a white background with a black frame); and a digital imaging device and digital processor (comprising for example a PDA or other handheld computer with built in or attachable camera).

The method associated with the system described includes initially tracing the perimeter of the wound on a transparent or translucent film in a manner already known to most clinicians in the field. Rather than re-tracing the outline a second time however, the transparency is positioned on a simple template that allows both scaling and off angle positioning in the imaging process. The digital imaging device of the system of the first preferred embodiment described above, captures the entire template with the wound trace contour inside. The digitized image is then processed by software within the unit to automatically find the template reference features and the trace and displays certain results on a display screen. The processing system of the present invention may also include the steps of image data thresholding, contour finding, square finding (used to identify the template), setting the region of interest (associated with the reference features on the template), wound trace finding, calculating the areas, eliminating distortion, and displaying the result with certain types of data filtering.

By using a digital camera or other imaging device it is possible to simplify the data input process and also to avoid errors caused by the manual second tracings associated with previous systems. The imagining method is also much more flexible than using a fixed-size touch sensitive pad. Positioning the wound trace in association with the template also makes the image processing much more reliable and accurate. The method of the present invention can further be used to measure multiple areas of a wound in a given region of tissue. The digital imaging device quality and processing power requirement of the system of the present invention are relatively low so the methodology can be embedded into a single simple microprocessor system. In contrast to the prior art, the present method uses a digital camera to capture the traced wound contour and then calculates the area by comparing it with the known size reference template features. There is no second tracing, and the wound size is only limited to the size of the template used. By changing the low cost template, the method can be used for any wound. The use of the template with reference features of known dimensions allows the imaging process to both scale the image and account for other than normal to the surface viewing angles.

A second preferred embodiment of the present invention consists simply of a digital imaging device (a digital camera, for example, 320×240 pixels or larger, color or black & white); and a processing unit (preferably a tablet PC or other microprocessor based computer system) having a touch-sensitive, display screen or other display associated means for providing graphical data input.

The method associated with the second preferred embodiment described above includes placing a small reference tag on the patient adjacent (but preferably outside) the wound and capturing a digital image of the wound site from a position generally normal to the plane of the wound area. The digital image is then transferred to a tablet PC or other computer having a display screen and a graphical data input device associated with the display screen (such as a touch sensitive panel). Preferably the display can be positioned for both viewing and for graphical data input. If the display is associated with a tablet PC, for example, it may be positioned to lay flat on a writing surface such as a desk. The image is then displayed on the screen of the PC and scaled (enlarged or reduced) to provide the clinician with an accurate view of the wound. The clinician then traces the wound perimeter with a stylus on the screen (or other type of graphical data input device) to define the extent of the wound. Software within the system then calculates the area of the wound based on the traced outline and the scale of the image (as referenced to the tag that is included in the field of view). Since the reference tag is designed to be easily recognizable to the computer the scaling can be very accurate. Defining the perimeter of the wound, on the other hand, is not so easy for the computer so this step in the process is left in the hands of the clinician.

In contrast to the prior art, this second embodiment, like the first, utilizes only a single tracing step and therefore greatly reduces the chance of introducing errors into the process. Unlike prior art methods that utilize touch pad technologies, the embodiment of the present invention described herein is able to beneficially scale the image of the wound before a trace is made by the clinician.

Also in contrast to much of the prior art, the methods of the present invention are simpler in terms of hardware requirements and set up as well as data processing requirements. In addition, many of the methods in the prior art do not work well on wounds that wrap around a limb or cannot otherwise by completely seen within a single picture frame.

The systems and methods of the present invention address most, if not all of the problems outlined above with the prior art systems. Ultimately, it is the present invention's use of the best aspects of other systems along with certain new elements, all in a new and unique way, which provides all of the advantages sought after in a single system.

### BRIEF DESCRIPTION OF THE DRAWINGS

FIG. 1 is a perspective view of the entire system of a first embodiment of the present invention shown in the progressive stages of the methodology of the invention.

FIG. 2 is a perspective view of the entire system of a second embodiment of the present invention shown in the progressive stages of the methodology of the invention.

FIG. 3A is a detailed view of a representative template utilized in conjunction with the first embodiment of the present invention showing a wound trace and distinguishing the various geometric measurements made through the imaging process of the present invention.

FIG. 3B is a detailed view of a PDA type device screen having captured an image of the representative template shown in FIG. 3A, again showing the wound trace and the various measurements made and used in the analysis of the wound area.

FIG. 4 is a “screen shot” view of a representative display generated by the system of the present invention showing the tracked progress of a healing wound.

FIG. 5 is a detailed view of a second representative template utilized in conjunction with the first embodiment of the present invention showing a wound trace involving multiple discrete wound beds.

FIG. 6A is a high level flow chart diagram showing the initial steps for implementation of the methodology of the first embodiment of the present invention.

FIG. 6B is a high level flow chart diagram showing the image processing steps of the methodology of the first preferred embodiment of the present invention.

FIG. 7A is a high level flow chart diagram showing the initial steps for implementation of the methodology of the second embodiment of the present invention.

FIG. 7B is a high level flow chart diagram showing the image processing steps of the methodology of the second preferred embodiment of the present invention.

### DETAILED DESCRIPTION OF THE PREFERRED EMBODIMENTS

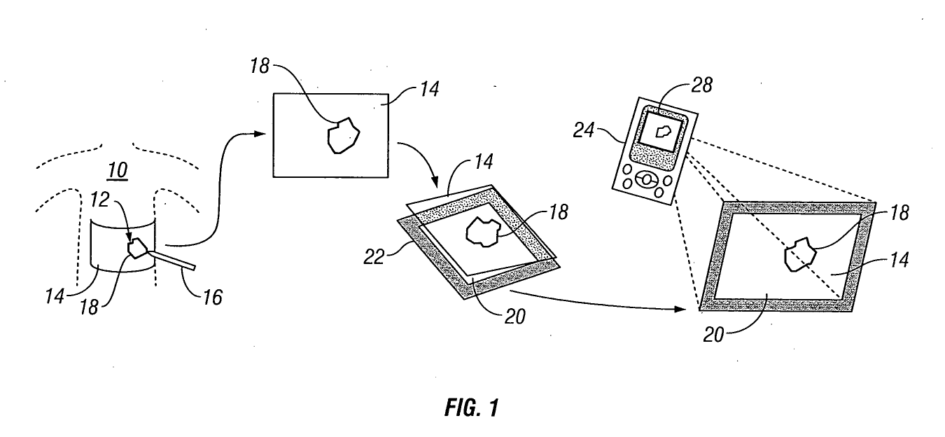

Reference is first made to FIG. 1 for a brief description of the specific components required within the system of the first preferred embodiment for implementing the methodology of the invention. In general the system involves the use of a transparent or translucent film positioned on the patient over the wound site onto which an outline trace of the wound perimeter is made with a permanent felt tip pen or the like. This transparent or translucent film bearing the wound trace is then positioned on a rectangular template frame, which in the preferred embodiment comprises a white background surrounded by a wide black band (frame). A clinician then uses a preprogrammed handheld digital processor and digital camera device (a PDA fitted with a camera, for example) to capture an image of the film/template assembly. Processing software programmed in the device identifies and quantifies the wound trace and the surrounding frame (as a reference) in order to calculate a wound area. This first method finds particular application in conjunction with wounds that extend over a larger, non-planar portion of the body, such as might be found with arm or leg wounds.

In referring to FIG. 1, all of the components of the system of the present invention are disclosed, as well as the progressive use of each of the components in carrying out the methodology of the present invention. In FIG. 1, patient 10 bearing wound 12 is shown with transparent/translucent film 14 carefully placed over wound 12 in order to establish a wound trace. The caregiver/clinician utilizes a felt tip pen 16 or other soft tip marking device, to gently trace an outline of the wound on transparent/translucent film 14, which results in wound trace 18 being permanently (or semi-permanently) fixed on transparent/translucent film 14.

Transparent/translucent film 14 is, of course, preferably sterile on at least the side placed against the wound. A variety of transparent, semi-transparent, or translucent sheet materials are available that comprise a removable backing that maintains an interior face of the sheet in a sterile condition until used. It has been found that for wounds undergoing reduced pressure treatment, the packaging associated with the layer of filter/foam (that is cut and placed in the wound bed) provides a suitable sterile transparent/translucent sheet material for use as the tracing medium. This packaging typically seals the filter/foam material between an opaque or translucent sheet and a transparent film. The interior faces of these sheets are, of course sterile until the package is opened which is typically accomplished by pulling the two sheets apart. If used immediately upon opening, the transparent sheet finds suitable application in the system of the present invention as the medium for tracing the wound outline.

Transparent/translucent film 14, which in the preferred embodiment may additionally bear some patient identification information, is then positioned on and fixed to backboard 20 to provide the image template assembly utilized in the system. Backboard 20 generally comprises a rigid or half-rigid board with a non-glossy surface bordered by frame 22 of a contrasting color. The contrasting color frame 22 may be any of a number of different types of frames suitable for creating an associated or enclosing, contrasting boundary for backboard 20. In the preferred embodiment, backboard 20 may be a non-glossy white or light color for example, and frame 22 may simply be a printed or painted black or dark color ink border that is also non-glossy. A physically separate frame of a contrasting color, into which the film is inserted, may also be used.

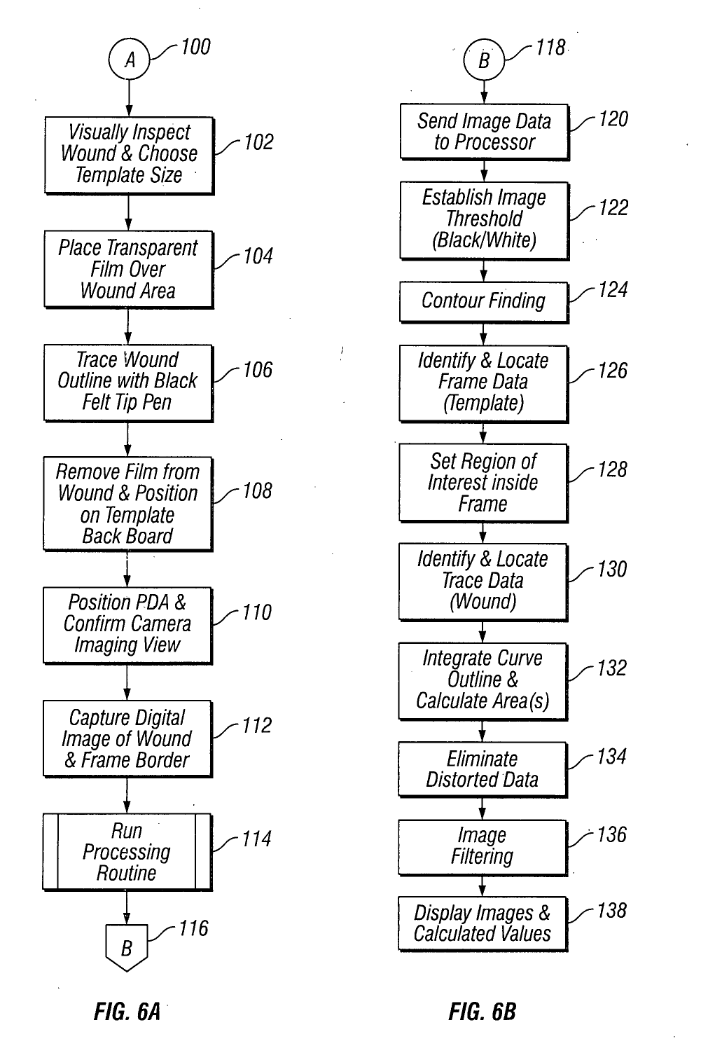

Once transparent/translucent film 14 is fixed to backboard 20, the assembly is placed in a convenient imaging position that provides a suitable presentation of the assembly to a digital camera connected with PDA device 24. A digital image 28 is created by the digital camera associated with PDA device 24 of the assembly of transparent/translucent film 14 and backboard 20. This digital image 28 is preferably viewed on the PDA device 24 in the process of capturing the still image in order to assure a complete image of the wound trace 18 and at least the interior border of frame 22. Once an appropriate image is captured, processing software operable within the microprocessor associated with PDA device 24 analyzes and quantifies the image data to return a value for the wound area. The methods for processing the image data and determining an area value are described in more detail below with respect to FIGS. 6A & 6B. In the preferred embodiment, the microprocessor system of PDA device 24 should be capable of handling modest amounts of digital image data and the associated processing requirements described below. Such processing requirements are minimal in nature and are generally fulfilled by standard handheld PCs, many of the latest PDAs and other handheld computing devices.

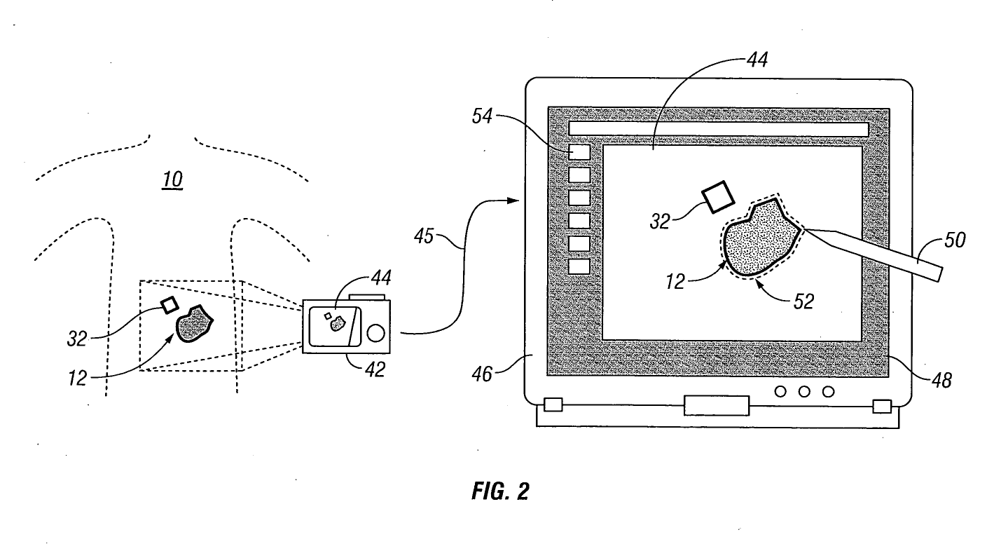

Reference is now made to FIG. 2 for a description of an alternate preferred embodiment of the present invention. Like the first embodiment, the second embodiment utilizes a single wound tracing action and the capturing of the wound trace in a digital image processing system. The difference is in the location of carrying out the wound trace. The system of the second preferred embodiment of the present invention consists simply of a digital imaging device (a digital camera); a processing unit such as a tablet PC or other microprocessor based computer system having a touch-sensitive, horizontally positionable, display screen (or an alternate method for inputting graphical information); and a stylus (or other user manipulable device) for directing the acquisition of data on the display screen.

In FIG. 2, patient 10 bearing wound 12 is shown positioned appropriately to have wound 12 imaged by digital imaging device 42. Reference tag 32 is placed adjacent (but preferably outside) the perimeter of wound 12 and is thereby also captured within a digital image 44 of the wound site taken by digital imaging device 42 from a position generally normal to the plane of the wound 12. The digital image 44 thus captured is then transferred to tablet PC 46 or other computer having a touch sensitive display screen 48 (preferably a display that can lay flat on a writing surface such as a desk). Transfer of digital image 44 over communication link 45 to tablet PC 46 may be by any of a number of different data communication protocols such as hardwire serial communication (USB for example) or wireless communication (such as IR or RF based protocols).

Image 44 is received into processing software operable within tablet PC 46 and is displayed on the screen of the tablet PC. The image here may readily be scaled (enlarged or reduced) to provide the clinician with an accurate and clear view of the wound 12. Various modifications to the image including scaling and contrast effects may be carried out by the clinician through function “keys” 54 displayed on display 48 in conjunction with image 44. The effort in this process, which is described in more detail below, is to provide the clinician with the best view of the wound to effect a perimeter trace that is accurate and consistent.

The clinician then traces the wound perimeter 52 with a stylus 50 on the screen 48 to define the extent of the wound 12. Alternate methods of graphical data input may be used in place of the touch screen display. Software within the system receives this data from the touch screen and establishes the scaled dimensions of the trace according to the methods described below. The trace provides the hard data that the processor may use to calculate the wound area without relying on the processor to make decisions regarding the true line defining the wound perimeter. This judgment step is left to the clinician. The reference tag 32 on the other hand is specifically designed to be easily recognizable to the image processor for the purpose of accurately determining the scale of the image. With the data associated with the trace and the reference tag image, the processor system within tablet PC 46 may then calculate the area of the wound and report it to the clinician on the display.

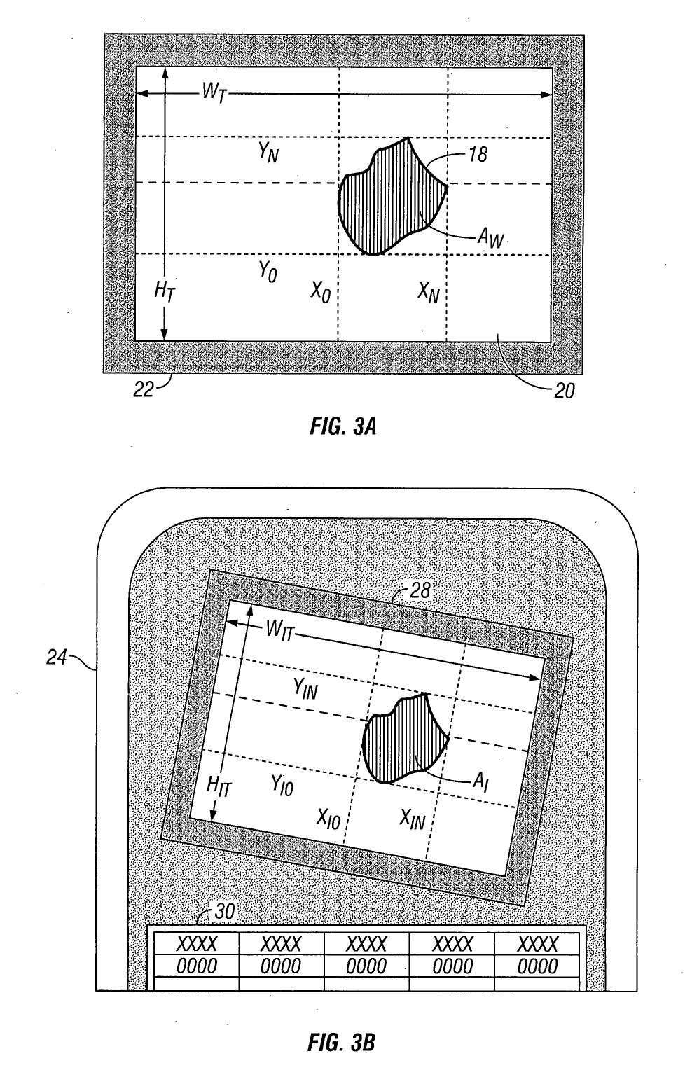

Reference is now made to FIG. 3A for a description of the two dimensional image acquired by the present invention in the first preferred embodiment and the various parameters within the image that are utilized in the processing of the data. FIG. 3A shows a typical image as acquired by the system, including an image of the frame 22 positioned on backboard 20. Wound trace 18 is shown fully contained within the area defined by frame 22.

Wound trace 18 encloses an area A_{W} that is the objective measurement of the system of the present invention. In order to obtain this measurement A_{W} the data associated with the image must be quantified in a manner that allows integration of the data and establishment of the area under (within) the curves associated with the wound trace. Various algorithms are known in the art for determining the area within a closed curve whose perimeter is established by known data points within a digitized field. In this case, the information necessary to carry out these calculations would include the overall width of the field, W_{T}, which is of course the width of the region of interest within the frame. Also necessary for such calculations is the height of the field, H_{T}, which likewise is defined by the dimensions of the frame. In each case, these two dimensions associated with the frame are known in actual size such that they become the reference dimensions for the actual wound area calculated. In this manner, the process of carrying out more complex calculations to eliminate off angle and three dimensional effects of the imaging process is made unnecessary. In other words, the actual size of the wound trace in the image is less important than its relative size with respect to the region of interest established by dimensions W_{T} and H_{T}.

Establishing the region of interest essentially establishes a coordinate field within which wound trace 18 is positioned. This coordinate field may therefore be analyzed as comprising curves in an X-Y coordinate frame with a minimum X value, X₀ extending to a maximum X value, X_{N} being the horizontal limits of the closed wound trace curve. Likewise, vertical minimums (Y₀) and maximums (Y_{N}) can be identified and established prior to digitally identifying coordinate ordered pairs for each of a number of selected points on the curve of the wound trace. Once again, techniques associated with both identifying points on a curve within a coordinate system and integrating those points to determine an area within the curve, are known in the art.

FIG. 3B provides a view of image 28 as might be presented on PDA device 24 as described above in conjunction with the first preferred embodiment of the present invention. In this view, which may be either during or after image capture, the template 20 with frame 22 is seen positioned at an obvious angle for emphasizing the capabilities of the system and method herein. Although the clinician may preferably hold the digital imaging device (the PDA device) in a position generally normal to the plane of the template 20 this positioning is not critical. As long as the entire interior edge of the frame 22 is captured in the image the process can determine the actual wound area.

In the view shown on the PDA device 24, wound trace 18 encloses an area A_{I} that is the scaled measurement of the true wound area. In order to obtain the actual value A_{W}, the data associated with the image must be scaled in both X and Y dimensions. In this case, the dimension W_{IT}, which is of course the image width of the region of interest within the frame 22. Likewise, the image height of the field, H_{IT}, which likewise is defined by the dimensions of the frame 22 are each independently compared to W_{IT} and H_{IT} to establish the scaling factors in each of the two dimensions. These scaling factors are then applied to the data coordinates representing the wound trace 18 to provide accurate values for the image X values, X_{I0} and X_{IN} being the horizontal limits of the closed wound trace curve, and Y_{I0} and Y_{IN}. Once again, techniques associated with both identifying points on a curve within a coordinate system and integrating those points to determine an area within the curve, are known in the art. The scaled data and the resultant calculation may then be displayed on the PDA device 24 in numerical form, in table 30 for example.

It is anticipated that a number of various enhancements to the systems (described above) and methodologies (described in more detail below) of the present invention could be made. Some of these enhancements are outlined immediately below, while others should be apparent to those skilled in the art.

### Providing Historical Imaging Display

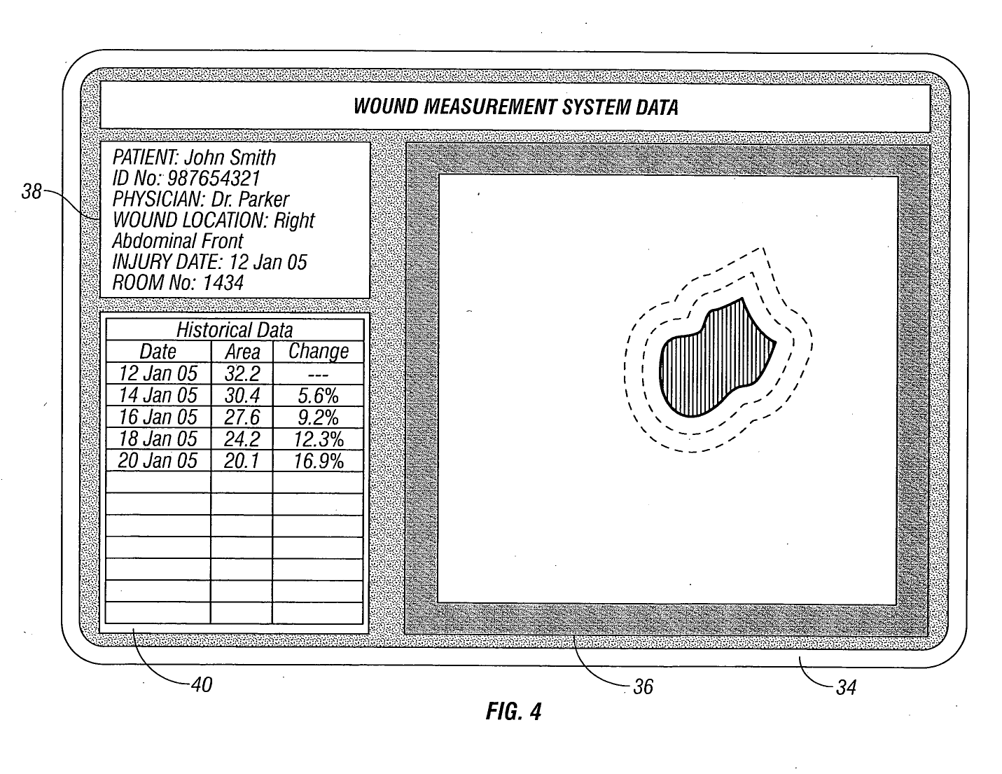

In addition to providing information on the changes in the absolute value of the wound area, (as described with wound data display 40 in FIG. 4 discussed below) it would be possible and desirable in some circumstances to actually provide an overlaid display that incorporates not only the current wound trace, but previous traces associated with the particular wound for a specific patient. In FIG. 4, these historical wound traces are shown in dashed or broken outline form in a manner that would not only allow the caregiver to identify the rate at which healing is occurring, but also identify certain areas of the wound that may be healing faster than others. Additional data storage in the processor system of the present invention is all that is required to carry out this enhancement.

### Discrimination of Wound Healing Zones

In the step of tracing the wound on the patient or on the screen image of the wound as described above, the caregiver or technician will typically outline what is most easily identifiable as the boundary of the wound, namely that line where traumatized or disrupted tissue meets stable or undisrupted skin tissue on the patient. Those skilled in the art will recognize, however, that there are often discernable zones of healing within a wound that might likewise be traced using the transparent/translucent film and dark felt tip pen elements of the first embodiment of the present invention or the touch screen display and stylus in the second embodiment described. Examples of such areas that may be of interest over time in discerning the progress in the healing of a wound include (from the outer periphery of the wound towards its interior) an area of reddening around the wound periphery associated with intact skin tissue, an area of initial granulation that typically defines the peripheral extent of the wound itself, and finally a serous zone in the interior of the wound wherein fluids may continue to exude during the healing process.

The identification of these various zones within the wound may permit the technician or healthcare provider to create a plurality of different traces, each corresponding to specific areas of interest. For example, the most interior of the closed curves would be the serous zone as defined by a small interior trace associated with the wound. Two closed curves surrounding the serous zone would identify the initial granulation zone or band by its interior extent and its exterior extent. Typically the exterior extent of the initial granulation zone would provide the overall boundary for the wound trace that is undefined by more specific zones of healing. Finally, a fourth trace, exterior to both the serous zone trace and the two initial granulation zone traces, could describe the area of reddening about the wound itself. Each of these areas could provide the healthcare provider relevant information about the healing process, and as a result provide guidance in the development of additional or continued regimens of treatment. While the above example illustrates one way in which multiple trace areas may be utilized, it is expected that the caregivers will determine their own particular scheme to best utilize this multi-area calculation capability. It would of course be important that each of these traces be closed curves in order for the digital image processor to accurately identify the area within any one of these curves.

Reference is now made again to FIG. 4 for a detailed description of the manner in which both calculated data and historical data may be displayed on a computer screen for viewing and analysis by the healthcare provider and/or the technician after processing by either of the two preferred embodiments of the present invention. In the first case the data may be stored and presented on the PDA device itself or may be uploaded to a larger system for later storage and viewing. Such uploading may occur through any of the various wired and wireless communication protocols established for such devices and may include Internet based communication protocols.

In FIG. 4, a typical screen shot is presented within data display 34. Data display 34 is comprised primarily of wound trace display 36, patient information display 38, and wound data information display 40. Wound trace display 36 is simply a recreation of the digital image acquired by the digital imaging devices in the processes of the present invention. Patient information display 38 is provided simply for the purposes of identifying and cataloging the wound data and the image data acquired. Although shown within the frame typically associated with the first preferred embodiment of the present invention, the display features described in FIG. 4 are equally applicable to the display of data acquired with the second preferred embodiment.

Wound data display 40 may provide not only the data associated with the current image established on the display, but may also provide historical data suitable for identifying changes in the character of the wound over time. Such information may, for example, include a wound area established at an initial measurement for a particular patient and a complete history of subsequent wound area measurements made on a periodic basis. In such a case, not only would the absolute value of the wound area be provided in this display, but percentage changes of this wound area may also be provided to allow the caregiver to more quickly discern the rate of healing that is occurring.

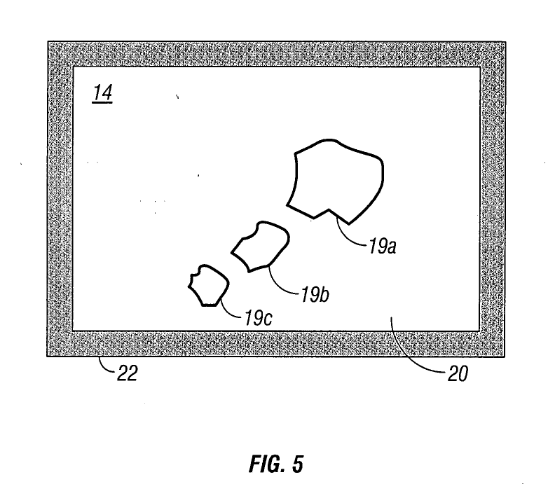

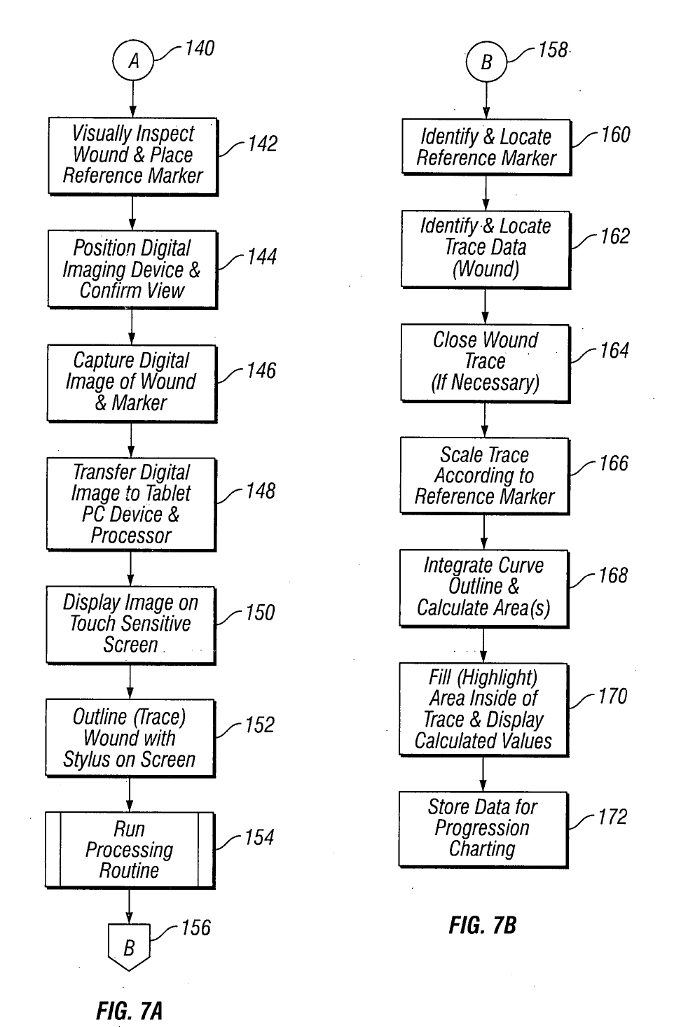

Reference is now made to FIG. 5 for a brief description of an alternate template usable in conjunction with the system of the present invention that comprises more than a single wound area. In this view, wound areas 19a, 19b, and 19c are shown as may be typical for many patients. The system and methodology of the present invention are entirely capable of identifying and dealing with multiple wound traces in the same manner. As the steps described above (and in more detail below) indicate, after the step of identifying a region of interest within a frame is carried out, the individual wound trace data is identified. This step (Step 130 in FIG. 6B and Step 162 in FIG. 7B below) may be repeated for any of a number of different wound traces that are discreetly identified within the region of interest. The only limitation on this process is the establishment of a wound trace within the boundaries of the frame (or associated with other types of reference areas) defining the template (in the case of the first preferred system) or within the field of the image (in the second preferred system). The process of digitizing and establishing these curves on a coordinate system is likewise simply a matter of progressing from one closed curve to the next in the calculation process.

The methods of the present invention follow from the preferred system embodiments described in detail above. For a discussion of the methods of the first preferred embodiment of the present invention, reference is now made to FIGS. 6A and 6B. These flowchart diagrams show the steps associated with acquiring (FIG. 6A) and processing (FIG. 6B) the wound trace data. FIG. 6A shows the initial process of acquiring a wound trace sufficient for digital processing. Image acquisition methodology 100 is initiated at Step 102 where the caregiver may visually inspect the wound and choose an appropriate template size to cover the wound. At Step 104, the caregiver places a transparent/translucent film over the wound area sufficient to cover all wound sections of concern. At Step 106, the caregiver or technician then traces a wound outline with a felt tip pen on the transparent/translucent film in a manner that places as little pressure on the wound surface as possible. The technician/caregiver then removes the film from the wound at Step 108 and positions the transparent/translucent film on the template backboard in a manner suitable for processing. In the preferred embodiment, the template backboard comprises a non-reflective white surface on a semi-rigid rectangular panel that is surrounded on its perimeter with a black, non-reflective frame as discussed above. Other colors and geometric shapes may be utilized for the background of the panel and the reference areas thereon.

Various mechanisms for adhering or fixing the transparent/translucent film to the backboard are contemplated. In a simplest of embodiments, the film may be taped at some part of its edge to the perimeter of the backboard in a manner that fixes it securely to prevent movement of the film with respect to the perimeter frame. More complex methods of affixing the film to the backboard could include the use of a rigid over-frame that may be positioned over the film on the backboard (such as with a picture frame). In any event, the objective is to simply prevent the movement of the wound trace with respect to the frame provided by the backboard during the imaging process.

At Step 110, the technician positions the PDA device (with its digital camera) to capture the entire view of the wound trace and at least the interior edge of the frame. Typically the digital camera utilized in the system of the present invention would provide an immediate imaging view (on the screen of the PDA device) that would allow the technician at Step 112 to confirm the proper view and thereafter trigger the digital camera to capture the image. The methodology of the present invention then enters, at Step 114, the image processing routine that is described in more detail below. The process flow chart is therefore continued at flow chart B by way of process connector 116.

FIG. 6B discloses in detail the various steps associated with the digital image processing of the wound trace image captured by the camera in the system of the present invention. Process 118 is initiated at Step 120 whereby the digital image is sent from the digital camera to the data processing components of the PDA device. Once again, in the preferred embodiment the requirements of the data processor are fulfilled by readily available handheld PC devices or PDA devices. Once the image data has been received by the processor, an initial establishment of the image threshold is carried out at Step 122. In this step the processor simply identifies the light (white) and dark (black) elements of the image and establishes a threshold value whereby an individual pixel on the image is identified as dark in contrast to the light background. The processor then carries out Step 124 of contour finding on the image, that is, establishing the data vectors that define the contours of the image.

Before proceeding to identify and process the wound trace, the processor identifies and locates the frame established on the template backboard at Step 126. The identification and location of the frame allow the processor, at Step 128, to set the region of interest as that area of the image as a whole that is inside the identified and located frame. In addition, the boundaries of the frame have a known geometry which therefore provides reference dimensions for accurately quantifying the wound size from the trace data.

Thereafter, at Step 130, the processor identifies and locates the trace data associated with the line image that was traced around the periphery of the wound. Once the data associated with the identified and located trace is established, mathematical processing associated with this data can be carried out. At Step 132, the processor carries out typical integration of the curve outline in order to calculate the area within the curve based on known geometric parameters associated with the identified frame and the set region of interest. Step 134 involves the elimination of distorted data based upon predetermined criteria intended to throw out clearly erroneous data often derived from distortions or errors in the imaging process. Finally at Step 136, various filtering procedures are carried out on the image to eliminate or reduce flickering lighting effects common with the imaging process.

After processing, the system of the present invention provides both an image display and references to the calculated values at Step 138. The character of the presentation of the data acquired and calculated, as well as the nature of the display, is as described above. In summary, the processing procedures of the first preferred method of the present invention include the following digital image processing steps; (1) an image thresholding process is carried out to allow discrimination between light and dark pixels in the image in a manner sufficient to characterize a pixel value as either empty or full (white or black); (2) an identification of the template square, which may typically be accomplished by associating it with the region on the periphery of the template, as well as identifying straight line edges to the rectangle; (3) a bracketing of the region of interest, namely inside the square; before (4) carrying out what is essentially a data scan of the pixel information contained within the bracketed region; and finally, in the process of examining the bracketed region, (5) the processor finds and identifies the wound tracing by distinguishing it from the empty or white background pixels.

Through a variety of algorithms known in the art, the processor may then assemble a closed curve of the wound tracing and calculate the area within the curve equating such with the area of the wound. Various data filtering methods may be utilized in the preferred embodiment, to remove distortion from the image and the data associated with the image before displaying the results on a computer display screen. A variety of other relevant patient information may be coordinated with the acquired wound healing information to provide the necessary tools for discerning the efficacy of the wound therapy and the need for possible modifications thereto.

For a discussion of the methods of the second preferred embodiment of the present invention, reference is now made to FIGS. 7A and 7B. These flowchart diagrams show the steps associated with acquiring (FIG. 7A) and processing (FIG. 7B) the wound trace data. FIG. 7A shows the initial process of acquiring the wound image and then a wound trace sufficient for digital processing. Image acquisition methodology 140 is initiated at Step 142 where the caregiver may visually inspect the wound and place an appropriate reference marker adjacent to or within the wound. At Step 144, the clinician positions the digital imaging device (the digital camera) and confirms that view covers wound sections of concern as well as the reference marker. At Step 146, the clinician then captures the digital image of the wound site with the digital imaging device. The technician/clinician then transfers the digital image data to the tablet PC device at Step 148 according to any of the various methods discussed above.

At Step 150, the technician views a display of the digital image of the wound site on the tablet PC and modifies various parameters associated with the image (scale, contrast, color, etc.) to clearly show the entire area of the wound and at the reference tag. The clinician then traces the wound perimeter (and any other closed areas of concern) with a stylus on the touch sensitive screen of the tablet PC device at Step 152. The methodology of the present invention then enters, at Step 154, the image processing routine that is described in more detail below. The process flow chart is therefore continued at flow chart B by way of process connector 156.

FIG. 7B discloses in detail the various steps associated with the digital image processing of the wound trace established by the clinician through the use of the stylus on the tablet PC touch screen display of the wound image. Process 158 is initiated at Step 160 whereby the reference marker is located within the digital image of the wound site. As discussed above, the reference tag is structured with a definitive outline border that is easily distinguished by contrasting pixels within the image data. This high contrast outline therefore provides the reference dimensions for scaling the image of the wound itself as calculations regarding the area of the wound are carried out.

Thereafter, at Step 162, the processor identifies and locates the trace data associated with the line that was traced by the clinician onto the touch screen of the tablet PC device, around the periphery of the wound. Preliminary to area calculations, the processing routine confirms the existence of closed curve traces and, at Step 164, closes the traces as accurately as possible. In the alternative, the process may notify the clinician that the traces established are not sufficient for processing to begin and request that they be re-established. Once the data associated with the identified and located trace is established, the data is scaled according to the known values for the reference marker. At Step 168, the processor carries out typical integration of the curve outline in order to calculate the area within the curve, again based on the known geometric scaling parameters associated with the identified and imaged reference tag. Step 170 involves presenting display information and features to highlight the area(s) of interest on the presented image of the wound and to report the calculated values both current and historical. Finally at Step 172, the data accumulated with the current image and calculated areas is stored for purposes of progressive charting and comparison with later measurements.

In summary, the processing procedures of the second preferred embodiment of the present invention include the following digital image processing steps; (1) a digital image of the wound site (with reference tag included) is acquired and communicated to a digital processing system incorporating a touch screen display; (2) an opportunity is provided to the clinician to improve the clarity of the image for the purpose of identifying the wound characteristics; (3) a tracing of the wound perimeter is made on the touch screen display thereby establishing a data set defining the wound perimeter; (4) reference is made to the acquired image of the reference tag to scale the data set defining the wound perimeter; and, through a variety of algorithms known in the art, the processor assembles a closed curve of the wound tracing data and calculates the area within the curve equating such with the area of the wound through ratio metric comparison to the included graphical frame or reference marker. As with the first preferred embodiment, highlighting of the image and otherwise displaying the results on a computer display screen convey the relevant information to the healthcare providers to establish, maintain, and/or modify a wound therapy regimen.

Although the present invention has been described in terms of the foregoing preferred embodiments, this description has been provided by way of explanation only, and is not intended to be construed as a limitation of the invention. Those skilled in the art will recognize modifications of the present invention that might accommodate specific patient and wound healing environments. Such modifications as to size, and even configuration, where such modifications are merely coincidental to the type of wound or to the type of therapy being applied, do not necessarily depart from the spirit and scope of the invention.

It is clear that the rectangular geometry of the template described above, for example, has been chosen primarily for its simplicity and those skilled in the art will recognize alternate geometries that achieve the same functionality as that of the rectangular frame described. It is also apparent that a tablet PC provides but one mechanism for allowing a clinician to establish a wound trace on a computer and that other methods, some of which may not involve a touch screen display, may provide the graphical data input required by the system of the present invention. References to black and white surface areas and transparent or translucent films, are meant to be exemplary only and not limiting of the types of materials that might be used with the various components of the system of the present invention.

We claim:

1. A system for determining and tracking the area of a wound, the system comprising:
a digital imaging device generally positioned at a spaced distance and angle from said wound, for acquiring a digital image of said wound and an area immediately surrounding said wound;
a reference tag removably positionable in association with said wound, said reference tag having discernable elements of known dimensions; and
a digital image display and processing device in data communication with said digital imaging device for receiving, displaying, and processing said acquired digital image, said display further comprising a graphical data input device for inputting data associated with a trace of said wound while said digital image is displayed thereon, said digital image display and processing device calculating and reporting a wound area based on said image and trace data.

2. The system of claim 1 wherein said digital imaging device comprises a digital camera having a data communications port for transferring digital image data from said digital camera to said digital image display and processing device.

3. The system of claim 1 wherein said reference tag comprises a flat geometric shape having a background and at least two orthogonal elements visually contrasting with said background, the orthogonal elements having known dimensions.

4. The system of claim 1 wherein said reference tag is disposable.

5. The system of claim 1 wherein said digital image display and processing device comprises a personal computer (PC), said PC comprising:
a data communication port for receiving said acquired digital image data from said digital imaging device;
an image display screen for displaying said acquired digital image received from said digital imaging device;
a graphical data input device operable in association with said image display screen for receiving data input from a user manipulable device, said graphical data input device for inputting data representing a trace of said wound as displayed on said image display screen; and
a nicroprocessor for processing said acquired digital image data and said wound trace data to calculate said area of said wound.

6. The system of claim 5 wherein said personal computer (PC) comprises a tablet PC and said image display screen is positionable in a plane generally suitable to both present a display of said digital image and operate said graphical data input device in association therewith.

7. The system of claim 5 wherein said personal computer (PC) further comprises digital storage for retaining said acquired digital image, said wound trace data, and said calculated area data.

8. The system of claim 5 wherein said personal computer (PC) further comprises a data communications system for transferring said acquired digital image, said wound trace data, and said calculated area data to a remote processing system.

9. The system of claim 5 wherein said personal computer (PC) further comprises programming for selectively modifying an appearance of said acquired digital image presented on said display, wherein such display modification improves definition of said wound.

10. The system of claim 1 further comprising a data communication cable connecting said digital imaging device and said digital image display and processing device for communicating said acquired digital image there between.

11. The system of claim 1 wherein said digital imaging device and said digital image display and processing device each further comprise wireless data communication systems for wirelessly communicating said acquired digital image there between.

12. A method for determining and tracking the area of a wound, the method comprising the steps of:
removably positioning a reference tag in an area immediately associated with said wound, said reference tag having discernable elements of known dimensions;
positioning a digital imaging device generally at a spaced distance and angle from said wound;
acquiring a digital image of said wound and said area immediately associated with said wound;
transferring data representing said acquired digital image from said digital imaging device to a digital image display and processing device, said digital image display and processing device having a display and a graphical data input device;
displaying said acquired digital image on said display device;
tracing at least a portion of said acquired digital image of said wound on said display with said graphical data input device, and thereby inputting trace data, while said acquired digital image is displayed on said display; and
calculating and reporting a wound area based on said acquired digital image and said input trace data.

13. The method of claim 12 further comprising the step of selectively modifying an appearance of said acquired digital image presented on said display, wherein such display modification improves definition of said wound.

14. The method of claim 12 further comprising the step of storing said acquired digital image, said wound trace data, and said calculated area data.

15. The method of claim 12 wherein said step of calculating and reporting a wound area comprises the steps of
generating a two dimensional trace data array representing the spatial locations of data input from said step of tracing said acquired digital image of said wound;
establishing an image threshold level between light and dark pixels within said acquired digital image;
identifying and locating digital image data associated with said reference tag positioned in association with said wound;
scaling said trace data array in at least two orthogonal spatial dimensions according to known actual dimensional values for said reference tag;
carrying out integration functions on said trace data array to calculate an area within the boundaries of said wound tracing; and
displaying the value of said calculated area within the boundaries of said wound tracing.

16. The method of claim 15 further comprising the step of displaying said wound tracing on said display of said digital image display and processing device, as said tracing is made, and monochromatically filling in an interior area of said tracing upon completion of said tracing into a closed curve.

17. The method of claim 14 further comprising repeating each of said steps periodically over time and tracking changes in said acquired digital image and said calculated area data.

18. The method of claim 17 further comprising the step of displaying the calculated values of said area within said wound tracing and said tracked changes over time on a visual display.

19. The method of claim 18 further comprising the step of concentrically displaying overlaid images of said wound tracings acquired over time on said visual display.

20. The method of claim 12 wherein said step of tracing at least a portion of said acquired digital image of said wound comprises tracing a closed outline of the image of a peripheral edge of the area of disrupted tissue associated with the wound.

21. The method of claim 12 wherein said step of tracing at least a portion of said acquired digital image of said wound comprises tracing a plurality of closed outlines of images of at least two zones of physiological character within the wound.

22. A method for determining and tracking the area of a wound, the method comprising the steps of:
removably positioning a reference tag in an area immediately associated with said wound, said reference tag having discernable elements of known dimensions;
positioning a digital imaging device generally at a spaced distance and angle from said wound;
acquiring a digital image of said wound and said area immediately associated with said wound;
transferring data representing said acquired digital image from said digital imaging device to a digital image display and processing device, said digital image display and processing device having a display and a graphical data input device;
displaying said acquired digital image on said display;
tracing at least a portion of said acquired digital image of said wound on said display with said graphical data input device, thereby inputting trace data, while said acquired digital image is displayed thereon;
generating a two dimensional trace data array representing the spatial locations of data input from said step of tracing said acquired digital image of said wound;
establishing an image threshold level between light and dark pixels within said acquired digital image;
identifying and locating digital image data associated with said reference tag positioned in association with said wound;
scaling said trace data array in at least two orthogonal spatial dimensions according to known actual dimensional values for said reference tag;
carrying out integration functions on said trace data array to calculate an area within the boundaries of said wound tracing; and
displaying the value of said calculated area within the boundaries of said wound tracing.
